(19) United States
(12) Patent Application Publication
(10) Pub. No.: US 2005/0273095 A1
(43) Pub. Date: Dec. 8, 2005

(54) Ablation catheters having anchoring capability and methods of using same

(75) Inventors: Miriam H. Taimisto, San Jose, CA (US); Jonathan A. Wohlgemuth, Morgan Hill, CA (US); Raj Subramaniam, Fremont, CA (US)

Correspondence Address: Bingham McCuthen, LLP, Suite 1800, Three Embarcadero, San Francisco, CA, 94111-4067, US

(73) Assignee: Scimed Life Systems, Inc., Maple Grove, MN (US)

(21) Appl. No.: 10/863,375

(22) Filed: Jun. 7, 2004

(51) Int. Cl. A61B 18/14

(52) U.S. Cl. 606/41

(57) ABSTRACT

A catheter includes a shaft having a distal end, an expandable member secured to the distal end, and an anchoring device positioned distal to the expandable member. The anchoring device having a delivery configuration and a deployed configuration. By way of one example, the anchoring device may comprise a shaped (e.g., helical) wire, the anchoring device having a cross-sectional dimension that allows it to secure itself inside a pulmonary vein when in its deployed configuration.

### FIELD OF THE INVENTION

The invention pertains to devices and methods for ablation of tissue, and more particularly, to ablation devices and methods for creating lesions within internal body organs, such as the heart.

### BACKGROUND

Physicians make use of catheters in medical procedures to gain access into interior regions of the body to ablate targeted tissue areas. For example, in electrophysiological therapy, tissue ablation is used to treat cardiac rhythm disturbances. During such procedures, a physician steers a catheter through a main vein or artery into an interior region of the heart. The physician positions an ablating element carried on the catheter near the targeted cardiac tissue, and directs energy from the ablating element to ablate the tissue, forming a lesion.

Such procedure may be used to treat arrhythmia, a condition in which abnormal electrical signals are generated in heart tissue. It has been shown that arrhythmias may be caused by ectopic focal points that are located immediately outside a pulmonary vein, in the area of an ostium. As such, when treating such as atrial fibrillation arrhythmias, it may be desirable to create a lesion at the ostium of a pulmonary vein. Such “extra-ostial” lesions can reduce a risk of pulmonary vein stenosis, and has been shown to provide a higher success rate in treating atrial fibrillation.

However, ablation of heart tissue poses a challenge in that the heart is constantly moving during an ablation procedure. As a result, it can be difficult to maintain stable contact between an ablating electrode and the target tissue, such as, e.g., tissue that is outside a pulmonary vein at the ostium.

### SUMMARY OF THE INVENTION

In an exemplary embodiment of the invention, an ablation catheter having a shaft with a proximal and distal ends, with an expandable member secured to the distal end of the shaft, is further provided with an anchoring device located distal to the expandable member. The anchoring device may be carried in a lumen of the catheter shaft, having a delivery configuration when inside the catheter lumen, and a deployed configuration when outside the lumen. In one embodiment, the anchoring device has a cross-sectional dimension that allows the anchoring device to secure itself inside a pulmonary vein when the anchoring device is deployed.

In accordance with a further aspect of the invention, a method for treating tissue in a body is provided, which includes securing an anchoring device inside a body cavity, placing an ablation assembly at an ostium of the body cavity, using the anchoring device to secure the ablation assembly relative to tissue at or adjacent the ostium of the body cavity, and using the ablation assembly to deliver ablation energy to the tissue.

Other and further aspects, embodiments and features of the invention will be evident from reading the following detailed description of the drawings, which is intended to illustrate, not limit, the invention.

### BRIEF DESCRIPTION OF THE DRAWINGS

Embodiments of the invention are illustrated by way of example, and not by way of limitation, in the figures of the accompanying drawings, in which like reference numerals refer to like components, and in which:

FIG. 1 illustrates an ablation system having an ablation catheter constructed in accordance with an exemplary embodiment of the invention;

FIG. 2A illustrates a distal end of the ablation catheter of FIG. 1, showing the ablation catheter having an ablation assembly and an anchoring device that are in their collapsed configurations;

FIG. 2B illustrates the distal end of the ablation catheter of FIG. 1, showing the ablation assembly and the anchoring device in their expanded configurations;

FIG. 3 illustrates a distal end of the ablation catheter of FIG. 1, showing the ablation assembly slidable relative to the anchoring device;

FIGS. 4A-4C illustrate a distal end of the ablation catheter of FIG. 1, showing the ablation catheter having a fluid channel connecting from the anchoring device to the ablation assembly;

FIG. 5 illustrates a distal end of an ablation catheter constructed in accordance with another exemplary embodiment of the invention, showing the ablation catheter having an expandable member;

FIG. 6 illustrates a variation of the expandable member of FIG. 5;

FIG. 7 illustrates a distal end of an ablation catheter having a guide wire lumen in accordance with another embodiment of the invention;

FIG. 8 illustrates a distal end of an ablation catheter having a steering wire in accordance with another embodiment of the invention;

FIGS. 9A-9E illustrate a exemplary method of using the ablation device of FIG. 1;

FIG. 10A illustrates a distal end of an ablation catheter having an anchoring device in accordance with another embodiment of the invention, showing the anchoring device in a delivery configuration;

FIG. 10B illustrates the distal end of the ablation catheter of FIG. 10A, showing the anchoring device in a deployed configuration;

FIG. 11A illustrates a distal end of an ablation catheter having an anchoring device in accordance with another embodiment of the invention, the anchoring device having a plurality of splines;

FIG. 11B illustrates a distal end of an ablation catheter having an anchoring device in accordance with yet another embodiment of the invention, showing the anchoring device having a fork configuration;

FIG. 11C illustrates a distal end of an ablation catheter having an anchoring device in accordance with still another embodiment of the invention, showing the anchoring device having a loop configuration;

FIG. 12 illustrates a distal end of an ablation catheter having an anchoring device in accordance with yet another embodiment of the invention, showing the anchoring device slidable relative to an ablation assembly.

### DESCRIPTION OF THE ILLUSTRATED EMBODIMENTS

Various embodiments of the invention are described hereinafter with reference to the figures. It should be noted that the figures are not drawn to scale and that elements of similar structures or functions are represented by like reference numerals throughout the figures. It should also be noted that the figures are only intended to facilitate the description of specific embodiments of the invention. They are not intended as an exhaustive description of the invention or as a limitation on its scope. In addition, an illustrated embodiment need not incorporate all possible aspects and features, and an aspect or feature shown or described in conjunction with one embodiment is not necessarily limited to that embodiment, but can be practiced in other embodiments of the invention, even if not so illustrated.

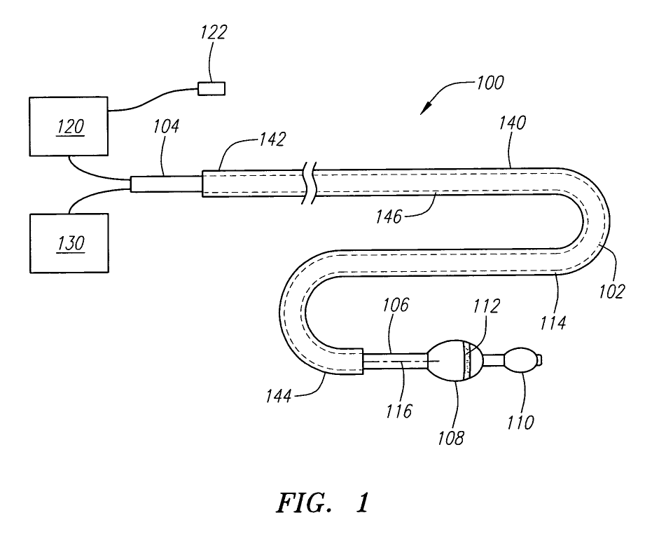

Referring to FIG. 1, a tissue ablation system 100 includes a sheath 140, an ablation catheter 102 slidable within the sheath 140, a ground electrode 122, a generator 120, and a pump 130. The catheter 102 includes a shaft 114 having a proximal end 104 configured for coupling to the generator 120 and the pump 130, and a distal end 106, to which an ablation assembly 108 and an anchoring device 110 are connected. The anchoring device 110 is configured to expand within a pulmonary vein during use, thereby securing the ablation assembly 108 relative to a target tissue at or adjacent an ostium. The ablation catheter 102 and the ground electrode 122 are electrically coupled to respective positive and negative terminals (not shown) of the generator 120, which is used for delivering ablation energy to the ablation assembly 108 to ablate target tissue. Particularly, the ablation assembly 108 has a conductive region 112 for making contact with a tissue and delivering ablation energy to the tissue. The generator 120 is preferably a radio frequency (RF) generator, such as the EPT-1000 XP generator available at Boston Scientific, Electrophysiology, San Jose, Calif. In some embodiments, either or both of the shaft 114 and the ablation assembly 108 may carry temperature sensor(s) (not shown) for sensing a temperature during use.

The sheath 140 has a proximal end 142, a distal end 144, and a lumen 146 extending between the proximal and the distal ends 142, 144. The lumen 146 is sized such that it could accommodate the ablation catheter 102 during use. In some embodiments, the sheath 140 can further include a steering mechanism for steering the distal end 144. The steering mechanism includes a steering wire having a distal end secured to the distal end 144 of the sheath 140, and a proximal end coupled to a handle, which includes a control for applying tension to the steering wire. Steering devices for catheters are well know in the art, and will not be described in further detail.

The shaft 114 has a circular cross-sectional shape and a cross-sectional dimension that is between 0.05 and 0.20 inch, and more preferably, between 0.066 and 0.131inch. However, the shaft 114 may also have other cross-sectional shapes and dimensions. The distal end 106 of the shaft 114 has a substantially pre-shaped rectilinear geometry. Alternatively, the distal end 106 may have a pre-shaped curvilinear geometry, which may be used to guide the anchoring device 110 away from a longitudinal axis 116 of the shaft 114. The shaft 114 can be made from a variety of materials, such as, a polymeric, electrically nonconductive material, like polyethylene, polyurethane, or PEBAX® material (polyurethane and nylon). Alternatively, the distal end 106 can be made softer than a proximal portion of the shaft 114 by using different material and/or having a thinner wall thickness. This has the benefit of reducing the risk of injury to tissue that the distal end 106 may come in contact with during a procedure.

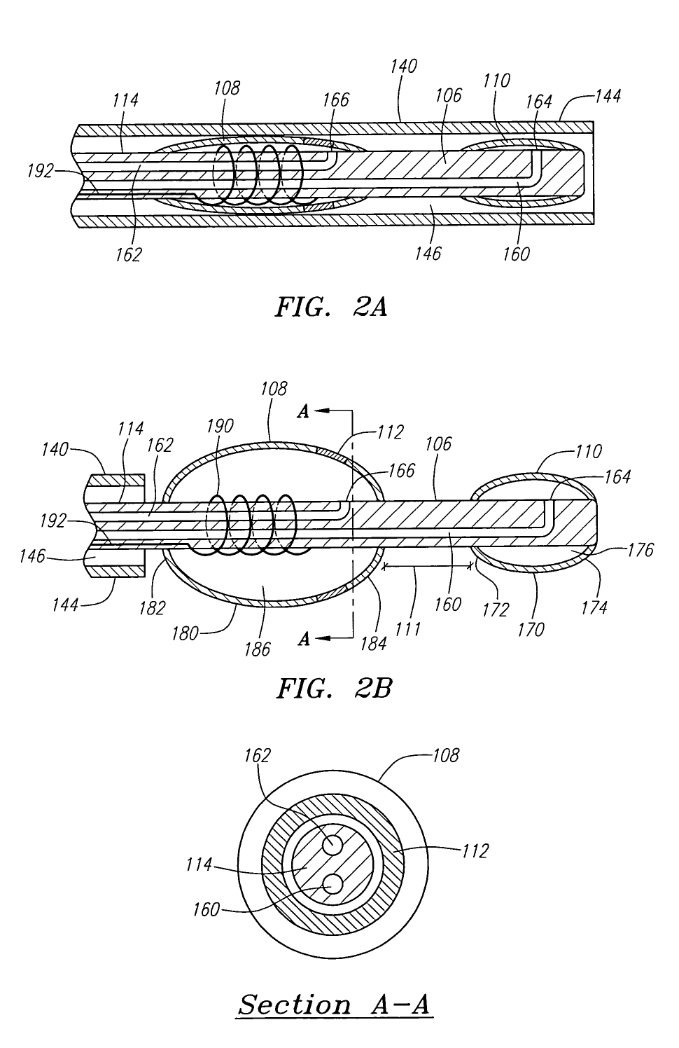

As shown in FIG. 2A, both the ablation assembly 108 and the anchoring device 110 are secured to a distal end 106 of the shaft 114, with the anchoring device 110 located distal to the ablation assembly 108. The anchoring device 110 and the ablation assembly 108 each has a collapsed (or delivery) configuration when resided within the lumen 146 of the sheath 140 (FIG. 2A). The anchoring device 110 and the ablation assembly 108 can each be expanded to have an expanded (or deployed) configuration when unrestricted outside the lumen 146 of the sheath 140 (FIG. 2B). In the illustrated embodiments, the anchoring device 110 is separated from the ablation assembly 108 by a distance 111 that is between 1-50 mm. Such configuration allows a pulmonary vein to conform to a shape of the anchoring device 110 when the anchoring device 110 is expanded in the pulmonary vein. Alternatively, the anchoring device 110 can be spaced at other distance from the ablation assembly 108. In other embodiments, the anchoring device 110 can abut against the ablation assembly 108.

In the illustrated embodiments, the anchoring device 110 includes an expandable-collapsible member 170, such as a balloon, having a proximal end 172 and a distal end 174 that are secured to the shaft 114. The expandable-collapsible member 170 can be made from a variety of materials, such as polymer, plastic, silicone, polyurethane, or latex. In some embodiments, the expandable-collapsible member 170 can be made from an elastic material such that the expandable-collapsible member 170 can stretch as it is being expanded. In other embodiments, the expandable-collapsible member 170 can be made from a non-stretchable material, which prevents the expandable-collapsible member 170 from stretching. In such cases, the expandable-collapsible member 170 is folded when it is in its collapsed configuration, and is unfolded as it is being expanded. The expandable-collapsible member 170 has a cross-sectional dimension that is between 10-35 mm, and more preferably, between 12-18 mm, when it is in the expanded configuration.

The expandable-collapsible member 170 can also have other cross-sectional dimensions as long as the expandable-collapsible member 170 can be secured within a body cavity, such as a pulmonary vein, after it has been expanded. In the illustrated embodiments, the expandable-collapsible member 170 has an elliptical shape, but can also have other shapes, such as a circular shape or a pear shape, in alternative embodiments. As shown in FIG. 2B, the shaft 114 includes a first port 164 in fluid communication with a first channel 160 for delivering fluid (gas or liquid) to a lumen 176 of the anchoring device 110. During use, fluid is conveyed under positive pressure by the pump 130, through the port 164 and into the lumen 176. The fluid fills the interior lumen 176 of the expandable-collapsible member 170, thereby exerting interior pressure that urges the expandable-collapsible member 170 from its collapsed geometry to its enlarged geometry. The first port 164 can also be used to drain delivered fluid from the lumen 176 to collapse the expandable-collapsible member 170.

The ablation assembly 108 includes an expandable-collapsible member 180, such as a balloon, having a proximal end 182 and a distal end 184 that are secured to the shaft 114. The expandable-collapsible member 180 can be made from a variety of materials, such as polymer, plastic, silicone, or polyurethane. In some embodiments, the expandable-collapsible member 180 can be made from an elastic material such that the expandable-collapsible member 180 can stretch as it is being expanded. In other embodiments, the expandable-collapsible member 180 can be made from a non-stretchable material, which prevents the expandable-collapsible member 180 from stretching. In such cases, the expandable-collapsible member 180 is folded when it is in its collapsed configuration, and is unfolded as it is being expanded. The expandable-collapsible member 180 has a cross-sectional dimension that is between 15-35 mm, and more preferably, between 20-30 mm, when it is in the expanded configuration.

The expandable-collapsible member 180 can also have other cross-sectional dimensions. In the illustrated embodiments, the expandable-collapsible member 180 has an elliptical shape, but can also have other shapes, such as a circular shape or a pear shape, in alternative embodiments. As shown in FIG. 2B, the shaft 114 includes a second port 166 in fluid communication with a second channel 162 for delivering a conductive fluid to a lumen 186 of the ablation assembly 108. During use, fluid is conveyed under positive pressure by the pump 130, through the second port 166 and into the lumen 186. The fluid fills the interior lumen 186 of the expandable-collapsible member 180, thereby exerting interior pressure that urges the expandable-collapsible member 180 from its collapsed geometry to its enlarged geometry. The second port 166 can also be used to drain delivered fluid from the lumen 186 to collapse the expandable-collapsible member 180. In the illustrated embodiments, the pump 130 has two reservoirs of fluid and two outlets for connecting to the channels 160, 162, and is configured to independently deliver fluid from the reservoirs to the anchoring device 110 and the ablation assembly 108 via the channels 160, 162, respectively. Alternatively, the pump 130 can have a single reservoir of fluid. In such cases, the channels 160, 162 are both connected to the reservoir, and fluid from the reservoir is used to inflate both the anchoring device 110 and the ablation assembly 108.

In some embodiments, either or both of the anchoring device 110 and the ablation assembly 108 can include, if desired, a normally open, yet collapsible, interior support structure to apply internal force to augment or replace the force of liquid medium pressure to maintain the member 170 (or member 180) in the expanded geometry. The form of the interior support structure can vary. It can, for example, comprise an assemblage of flexible spline elements, or an interior porous, interwoven mesh or an open porous foam structure. The interior support structure is located within the interior lumen 176 of the member 170 (or the interior lumen 186 of the member 180) and exerts an expansion force to the member 170 (or member 180) during use. Alternatively, the interior support structure can be embedded within a wall of the member 170 (or member 180).

The interior support structure can be made from a resilient, inert material, like nickel titanium (commercially available as Nitinol material), or from a resilient injection molded inert plastic or stainless steel. The interior support structure is preformed in a desired contour and assembled to form a desired support skeleton. In some embodiments, the anchoring device 110 and the ablation assembly 108 each has an interior support structure for urging the anchoring device 110 and the ablation assembly 108 to expand when they are un-confined outside the lumen 146 of the sheath 140. In such cases, the ablation system 100 does not include the pump 130, and the shaft 114 does not include the channels 160, 162.

In the illustrated embodiment, the conductive region 112 of the ablation assembly 108 has a ring configuration, but can have other shapes or configurations in alternative embodiments. The conductive region 112 is located distal to a proximal one-third of the member 180, and more preferably, located at a distal one-third of the member 180. However, in other embodiments, the conductive region 112 can be located at other positions as long as the conductive region 112 can make contact with a tissue desired to be ablated when the member 180 is in the expanded configuration. The conductive region 112 can be variously constructed. In some embodiments, the conductive region 112 of the ablation assembly 108 includes an electrically conducting shell that is disposed upon the exterior of the expandable-collapsible member 180. Preferably, the shell is not deposited on the proximal one-third surface of the member 180. This requires that the proximal surface of the member 180 be masked, so that no electrically conductive material is deposited there. This masking is desirable because the proximal region of the ablation assembly 108 is not normally in contact with tissue. The shell may be made from a variety of materials having high electrical conductivity, such as gold, platinum, and platinum/iridium. These materials are preferably deposited upon the unmasked, distal region of the member 180. Deposition processes that may be used include sputtering, vapor deposition, ion beam deposition, electroplating over a deposited seed layer, or a combination of these processes. In other embodiments, the shell comprises a thin sheet or foil of electrically conductive metal affixed to the wall of the member 180. Materials suitable for the foil include platinum, platinum/iridium, stainless steel, gold, or combinations or alloys of these materials. The foil preferably has a thickness of less than about 0.005 cm. The foil is affixed to the member 180 using an electrically insulating epoxy, adhesive, or the like.

In other embodiments, a portion of the expandable-collapsible wall forming the member 180 is extruded with an electrically conductive material to form the conductive region 112. Materials suitable for co-extrusion with the expandable-collapsible member 180 include carbon black and chopped carbon fiber. In this arrangement, the co-extruded portion of the expandable collapsible member 180 is electrically conductive. An additional shell of electrically conductive material can be electrically coupled to the co-extruded portion, to obtain the desired electrical and thermal conductive characteristics. The extra external shell can be eliminated, if the co-extruded member 180 itself possesses the desired electrical and thermal conductive characteristics. The amount of electrically conductive material co-extruded into a given member 180 affects the electrical conductivity, and thus the electrical resistivity of the member 180, which varies inversely with conductivity. Addition of more electrically conductive material increases electrical conductivity of the member 180, thereby reducing electrical resistivity of the member 180, and vice versa.

The above described expandable-collapsible bodies and other expandable structures that may be used to form the ablation assembly 108 are described in U.S. Pat. Nos. 5,846,239, 6,454,766 B1, and 5,925,038, which the entire disclosure of each is expressly incorporated by reference herein.

In the illustrated embodiments, the ablation catheter 102 also includes an electrode 190 that is secured to the shaft 1-14, and a wire 192 that is connected to the electrode 190 and is disposed within a wall of the shaft 114. The electrode 190 is composed of a material that has both a relatively high electrical conductivity. Materials possessing these characteristics include gold, platinum, platinum/iridium, among others. Noble metals are preferred. Alternatively, the electrode 190 can be made of electrically conducting material, like copper alloy or stainless steel. The electrically conducting material of the electrode 190 can be further coated with platinum-iridium or gold to improve its conductive properties and biocompatibility. In the illustrated embodiments, the electrode 190 includes a coil that is disposed coaxially outside the shaft 114. In alternative embodiments, the electrode 190 has a tubular shape and is disposed in a recess on an exterior surface of the shaft 114 such that the electrode 190 forms a substantially smooth surface with the exterior surface of the shaft 114. The electrode 190 can also have other shapes and configurations.

During use, the electrode 190 and the ground electrode 122 are electrically coupled to the generator 120, with the ground electrode 122 placed on a patient's skin. The generator 120 delivers a current to the electrode 190, and the conductive fluid within the lumen 186 of the expandable-collapsible member 180 conducts the current to the conductive region 112. In this case, ablation energy will flow from the conductive region 112 to the ground electrode 122, which completes a current path, thereby allowing tissue to be ablated in a mono-polar arrangement. Alternatively, the ablation catheter 102 additionally includes a return (or indifference) electrode, which allows tissue to be ablated in a bi-polar arrangement. In this case, ablation energy will flow from one electrode (the ablating electrode) on the catheter 102 to an adjacent electrode (the indifferent electrode) on the same catheter 102.

In other embodiments, instead of using the delivered fluid to conduct current from the electrode 190 to the conductive region 112, current is delivered from the generator 120 to the conductive region 112 via a RF wire. In such case, the ablation catheter 102 includes a RF wire that electrically connects the conductive region 112 to the generator 120. The RF wire may be embedded within the wall of the expandable-collapsible member 180, or alternatively, be carried within the interior lumen 186 of the expandable-collapsible member 180.

Also, in other embodiments, the ablation assembly 108 does not have the conductive region 112. In such cases, the member 180 comprises an electrically non-conductive thermoplastic or elastomeric material that contains the pores on at least a portion of its surface. The fluid used to fill the interior lumen 186 of the member 180 establishes an electrically conductive path, which conveys radio frequency energy from the electrode 190. The pores of the member 180 establish ionic transport of ablation energy from the internal electrode 190, through the electrically conductive medium, to tissue outside the member 180.

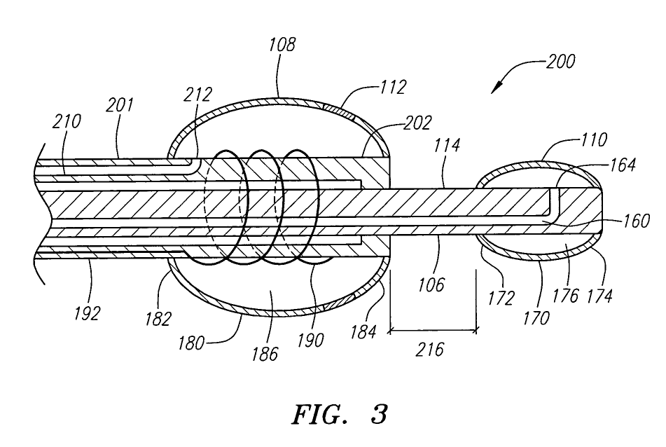

FIG. 3 shows an ablation catheter 200 that is similar to ablation catheter 102, except that the ablation assembly 108 is not secured to the shaft 114. In the illustrated embodiments, the ablation assembly 108 is secured to a distal end 202 of an outer tube 201, which is coaxially surrounding the shaft 114. The outer tube 201 is slidable relative to the shaft 114, thereby allowing a spacing 216 between the ablation assembly 108 and the anchoring device 110 be adjusted during use. The outer tube 201 includes a channel 210 terminating at a port 212 that is in communication with the lumen 186 of the ablation assembly 108. The channel 210 is used for delivering fluid to the lumen 186 of the ablation assembly 108 to expand the ablation assembly 108. The channel 210 can also be used to drain delivered fluid from the lumen 186 to collapse the ablation assembly 108, as similarly discussed previously.

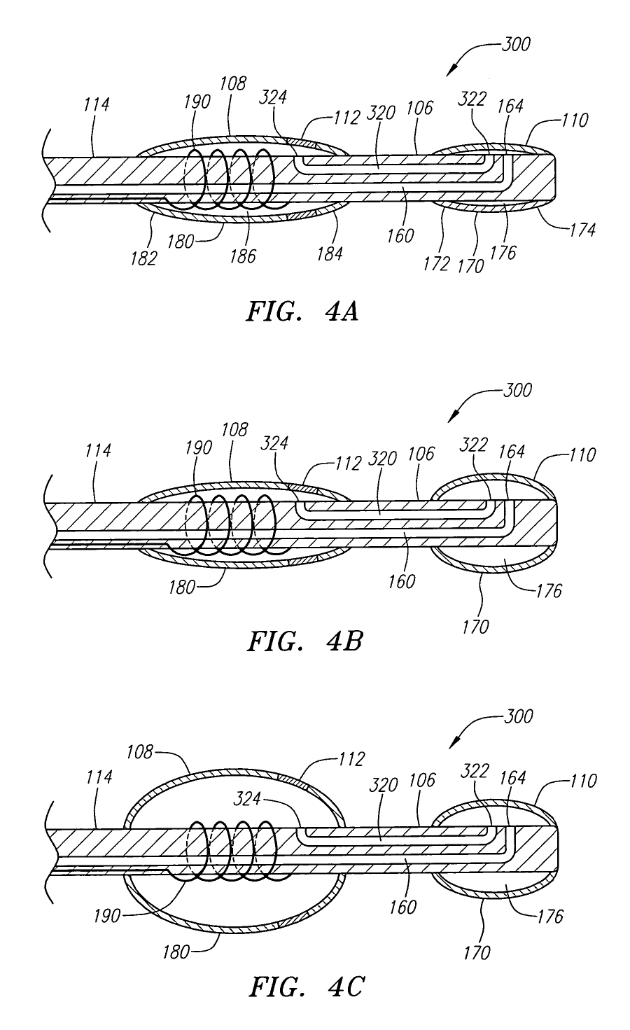

In the above described embodiments, separate channels extending from a proximal end to a distal end of the ablation device are used to deliver fluid to and from the ablation assembly 108 and the anchoring device 110. However, a single channel extending from a proximal end to a distal end of the ablation device can be used. FIGS. 4A-4C illustrate an ablation catheter 300, which is similar to the ablation device 102, except that the shaft 114 does not have the second channel 162. In such cases, the shaft 114 includes the first channel 160 for delivering fluid to the lumen 176 of the anchoring device 110, and a second channel 320 extending from the anchoring device 110 to the ablation assembly 108. During use, the pump 130 delivers inflation fluid to the anchoring device 110 via the first channel 160 to expand the anchoring device 110. Particularly, delivered fluid exits from the first port 164 and fills the lumen 176 of the expandable-collapsible member 170.

The delivered fluid inflates the expandable-collapsible member 170 until the expandable-collapsible member 170 can no longer expand, at which point, fluid delivered inside the lumen 176 will flow into a second port 322 and travel to the ablation assembly 108 via the second channel 320 (FIG. 4B). The fluid exits from a third port 324 and fills the lumen 186 of the expandable-collapsible member 180 to expand the ablation assembly 108 (FIG. 4C). As such, the ablation catheter 300 allows the anchoring device 110 be expanded before the ablation assembly 108. In other embodiments, check-valves can be secured to any or all of the ports 164, 322, 324 to ensure a flow direction of the fluid.

In other embodiments, instead of having the second channel 320 extending from the anchoring device 110 to the ablation assembly 108, the shaft 114 can include a channel that branches out from the first channel 160 and extends to the ablation assembly 108. Such configuration allows the expandable-collapsible members 170, 180 to be expanded substantially simultaneously. Also, in other embodiments, the expandable-collapsible members 170, 180 can be made from different materials, or have different wall thicknesses, thereby providing different expansion responses for the members 170, 180.

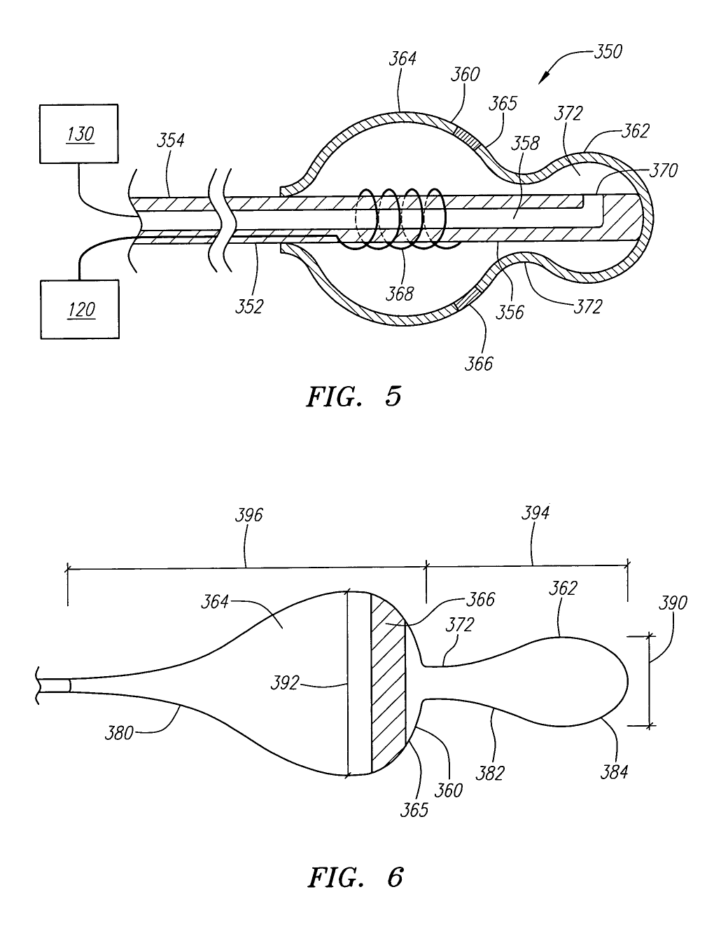

In the above-described embodiments, the ablation assembly 180 and the anchoring device 110 are separate components that are secured to the shaft 114. However, in alternative embodiments, the ablation assembly 180 can be manufactured with the anchoring device 110 as a single unit. FIG. 5 illustrates an ablation catheter 350, which includes a shaft 352 having a proximal end 354, a distal end 356, a channel 358 extending between the proximal and the distal ends 354, 356, and an electrode 368 secured to the shaft 352. In the illustrated embodiments, the electrode 368 has a helical shape, but can have different shapes and configurations in alternative embodiments. The shaft 352 has a port 370 at which the channel 358 terminates. In other embodiments, the port 370 can be located at other positions along the length of the shaft 352, and the ablation catheter 350 can have more than one ports. The ablation catheter 350 also includes an expandable-collapsible member 360 having a distal portion (anchor portion) 362 and a proximal portion (treatment portion) 364, and a conductive region 366 on the member 360.

In the illustrated embodiments, the conductive region 366 has a ring configuration and is located at a distal end 365 of the proximal portion 364. Alternatively, the conductive region 366 can have other shapes and can be located at other positions on the expandable-collapsible member 360. The distal portion 362 of the expandable-collapsible member 360 is configured to be inserted and expanded inside a body cavity, such as a pulmonary vein, thereby anchoring the proximal portion 364 relative to a tissue to be ablated. As such, the distal portion 362 should have a shape and a cross-sectional dimension that allow the distal portion 362 to be secured inside the cavity when the distal portion 362 is expanded. In the illustrated embodiments, the expandable-collapsible member 360 has a recess 372, which allows a pulmonary vein to conform to the shape of the distal portion 362 without distorting the ostium. In other embodiments, the expandable-collapsible member 360 does not have the recess 372.

During use, fluid is pumped into the channel 358 by the pump 130, and exits from the port 370 into a lumen 372 within the expandable-collapsible member 360, thereby expanding the expandable-collapsible member 366. The expandable-collapsible member 360 is configured such that the distal portion 362 is expanded before the proximal portion 364. For example, the distal portion 362 can be made from a material that is relatively more flexible or elastic than the proximal portion 364. Alternatively, the distal portion 362 can have a wall thickness that is relatively thinner than that of the proximal portion 364. More alternatively, stiffening member(s), such as wire(s), can be secured to the proximal portion 364, thereby stiffening the proximal portion 364. In other embodiments, the expandable-collapsible member 360 is configured such that the distal and the proximal portions 362, 364 expand simultaneously. After the proximal portion 364 has been expanded, the generator 120 delivers ablation energy to the electrode 368, and the fluid within the lumen 372 conducts the energy to the conductive region 366, thereby ablating tissue that is in contact with the conductive region 366.

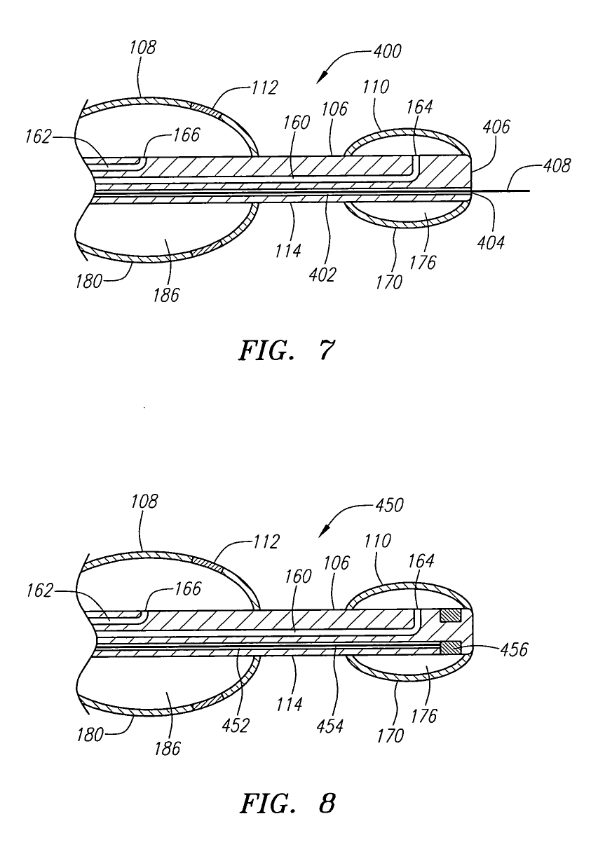

In other embodiments, the expandable-collapsible member 360 can have different shapes. FIG. 6 shows a variation of the expandable-collapsible member 360 having a shape that resembles an hourglass. In the illustrated embodiment, a proximal end 380 of the proximal portion 364 is relatively more tapered than the distal end 360, and a proximal end 382 of the distal portion 362 is relatively more tapered than a distal end 384. The distal portion 362 has a cross-sectional dimension 390 that is between 10-20 mm, and more preferably, between 12-18 mm, and the proximal portion 364 has a cross sectional dimension 392 that is between 15-35 mm, and more preferably, between, 20-30 mm. Also, the distal portion 362 has a length 394 that is between 10-20 mm, and more preferably, between 12-18 mm, and the proximal portion 364 has a length 396 that is between 15-70 mm, and more preferably, between 20-30 mm. In other embodiments, the expandable-collapsible member 360 can have other dimensions.

In any of the embodiments of the ablation catheter described herein, the shaft of the ablation catheter can further includes a guide wire lumen for accommodating a guide wire. FIG. 7 illustrates an ablation catheter 400 which includes a guide wire lumen. The ablation catheter 400 is similar to the ablation catheter 102, except that the shaft 114 further includes a lumen 402 extending from the proximal end 104 to the distal end 106. The lumen 402 terminates at a port 404 located at a distal tip 406 of the shaft 114. During use, the lumen 402 can be used to house a guide wire 408.

In any of the embodiments of the ablation catheter described herein, the ablation catheter can further include a steering mechanism for steering a distal end of the shaft. FIG. 8 illustrates an ablation catheter 450 that is similar to the ablation catheter 102 except that it further includes a lumen 452, a steering wire 454 disposed within the lumen 452, and a ring 456 for securing the steering wire 454 to the distal end 106 of the shaft 114. A proximal end of the steering wire 454 is connected to a steering mechanism (not shown) having a steering lever operable for steering the distal end 106 of the shaft 114. Particularly, the steering mechanism is configured to apply a tension to the steering wire 454, thereby bending the distal end 106 of the shaft 114 to. The steering mechanism can includes a locking lever operable in a first position to lock the steering lever in place, and in a second position to release the steering lever from a locked configuration. Further details regarding this and other types of handle assemblies can be found in U.S. Pat. Nos. 5,254,088, and 6,485,455 B1, the entire disclosures of which are hereby expressly incorporated by reference. In other embodiments, the steering wire 454 can be secured to the shaft 114 in other configurations. Also, in other embodiments, instead of having one steering wire 454, the ablation catheter 450 can include more than one steering wires for steering the distal end 106 of the shaft 114 in a plurality of directions.

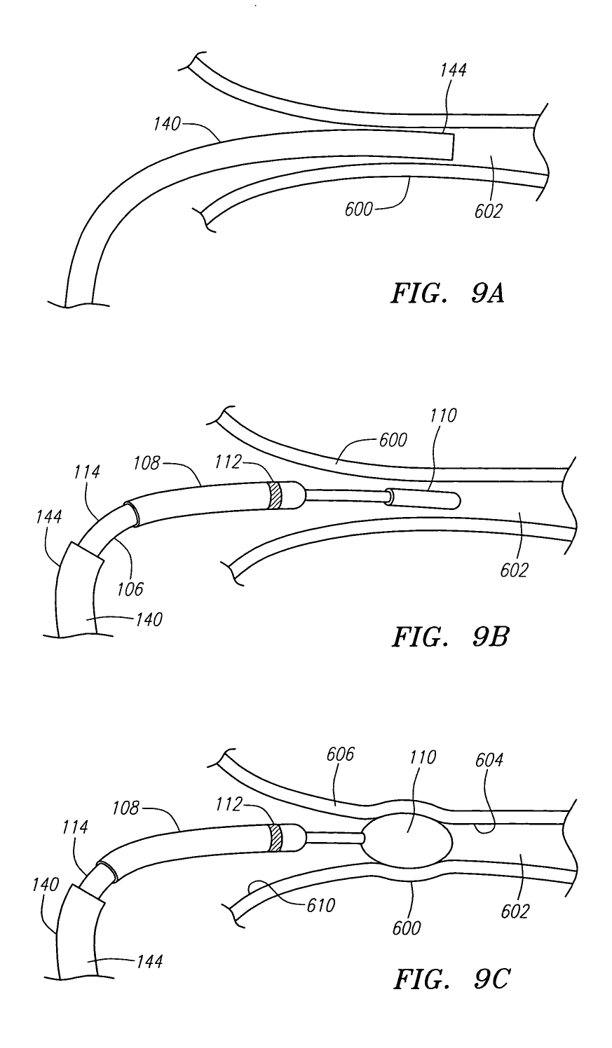

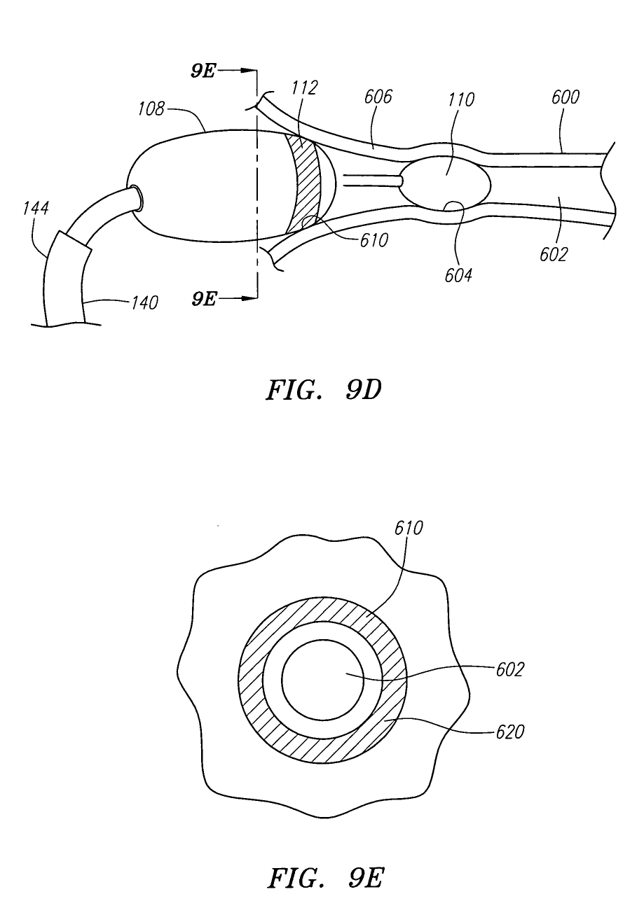

Refer to FIGS. 9A-9E, a method of using the system 100 will now be described with reference to cardiac ablation therapy. Particularly, the method will be described with reference to the embodiment of the ablation system 100 shown in FIG. 1. However, it should be understood by those skilled in the art that similar methods described herein may also apply to other embodiments of the system 100 previously described, or even embodiments not described herein.

When using the system 100 for cardiac ablation therapy, the sheath 140, using a dilator and a guidewire, is inserted through a main vein (typically the femoral vein), and is positioned into a right atrium of a heart using conventional techniques. Once the distal end 144 of the sheath 140 is placed into the atrium, the guidewire is then removed. Next, a needle can be inserted into the lumen 146 of the sheath 140 and exits from the distal end 144 to puncture an atrial septum that separates the right and left atria. Alternatively, the sheath 140 can have a sharp distal end 144 for puncturing the atrial septum, thereby obviating the need to use the needle. The distal end 144 of the sheath 140 (together with the dilator) is then advanced through the atrial septum, and into the left atrial chamber. Once at the left atrial chamber, the dilator is removed, and a guidewire, the catheter 102 (if it is steerable), or other steerable catheter or device, can be inserted into the lumen 146 of the sheath 140, and be used to steer the distal end 144 of the sheath 140 towards a lumen 602 of a pulmonary vein 600 (FIG. 9A). Alternatively, if the sheath 140 is steerable, it can be steered (e.g., using a steering mechanism) towards the lumen 602. The sheath 140 is then advanced distally until the distal end 144 is desirably placed inside (or adjacent) the lumen 602 of the pulmonary vein 600.

Next, if the catheter 102 was not used to steer the sheath 140, the catheter 102 is then inserted into the lumen 146 of the sheath 140. When the catheter 102 is inside the lumen 146, the ablation assembly 108 and the anchoring device 110 are confined within the lumen 146 in their collapsed configurations. The catheter 102 is advanced within the lumen 146 until the anchoring device 110 is at the distal end 144 of the sheath 140. The sheath 140 is then retracted relative to the ablation catheter 102, thereby exposing the anchoring device 110 in the pulmonary vein 600 (FIG. 9B). In the illustrated embodiments, the sheath 140 is retracted such that both the anchoring device 110 and the ablation assembly 108 are outside the sheath 140. If the ablation catheter 300 of FIG. 4 or the ablation catheter 350 of FIG. 5 is used, the sheath 140 can be retracted to expose only the anchoring device 110 and not the ablation assembly 108, thereby ensuring that the anchoring device 110 will be expanded before the ablation assembly 108. Alternatively, since the ablation catheter 300/350 is configured to have the anchoring device 110 expand before the ablation assembly 108, the sheath 140 can be retracted to deploy both the anchoring device 110 and the ablation assembly 108.

It should be noted that other methods can also be used to place the distal end of the catheter 102 into the lumen 602 of the pulmonary vein 600. For example, if the ablation catheter 102 has a guide wire lumen, such as that shown in FIG. 7, the guide wire 408 can be inserted through a separate cannula and into the lumen 602 of the pulmonary vein 600. The ablation catheter 102, together with the sheath 140, are then inserted into the cannula and over the guide wire 408, and are advanced into the lumen 602 of the pulmonary vein 600 using the guide wire 408 as a guide. Alternatively, if the ablation catheter 102 is steerable, such as that shown in FIG. 8, the ablation catheter 102 can be steered into the lumen 602 of the pulmonary vein 600 while it is housed within the lumen 146 of the sheath 140.

After the anchoring device 110 has been desirably positioned within the lumen 602 of the pulmonary vein 600, inflation fluid is delivered under positive pressure by the pump 130 to urges the anchoring device 110 to expand (FIG. 9C). The expanded anchoring device 110 exerts a pressure against an interior surface 604 of the pulmonary vein 600, thereby securing the anchoring device 110 relative to the pulmonary vein 600. Because of the pressure exerted by the anchoring device 110, the pulmonary vein 600 at the location of the anchoring device 110 is slightly enlarged. However, due to a separation between the anchoring device 110 and the ablation assembly 108, and/or a shape of the anchoring device 110, a portion 606 of the pulmonary vein 600 adjacent the ostium 610 is not stretched, and the shape of the ostium 610 is relatively unaffected by the anchoring device 110.

Next, ionic fluid is then delivered under positive pressure by the pump 130 to urge the ablation assembly 108 to expand (FIG. 9D). The expanded ablation assembly 108 causes the conductive region 112 to press against the ostium 610. If the ablation catheter 200 of FIG. 3 is used, the ablation assembly 108 can be positioned relative to the anchoring device 110 to make contact with the ostium 610 and/or to adjust a compressive pressure against the ostium 610, by advancing or retracting the outer tube 201 relative to the shaft 114. Because the ablation assembly 108 is secured relative to the ostium 610 by the anchoring device 110, the ablation assembly 108 is maintained contact with the ostium 610, which is constantly moving due to the beating heart.

Next, with the ablation catheter 102 coupled to the output port of the RF generator 120, and the ground electrode 122 coupled to the return/ground port of the RF generator 120, ablation energy is delivered from the generator 108 to the electrode 190 of the ablation catheter 102. Electric current is transmitted from the electrode 190 to the ions within the fluid that is inside the expandable-collapsible member 180. The ions within the fluid convey RF energy to the conductive region 112, which ablates the ostium tissue in a mono-polar arrangement (if the ground electrode 122 is used) or a bi-polar arrangement (if the ablation catheter 102 includes a return electrode). If the expandable-collapsible member 180 is porous, ions within the fluid convey RF energy through the pores into the target tissue and to the ground electrode 122, thereby ablating the ostium tissue.

After a lesion 620 has been created at the ostium 610 (FIG. 9E), the fluid is discharged to deflate the anchoring device 110 and the ablation assembly 108. If additional ostium(s) of other pulmonary vein(s) needs to be ablated, the above described steps can be repeated to create additional lesion(s). After all desired lesions have been created, the ablation catheter 102 and the sheath 140 are then retracted and removed from the interior of the patient.

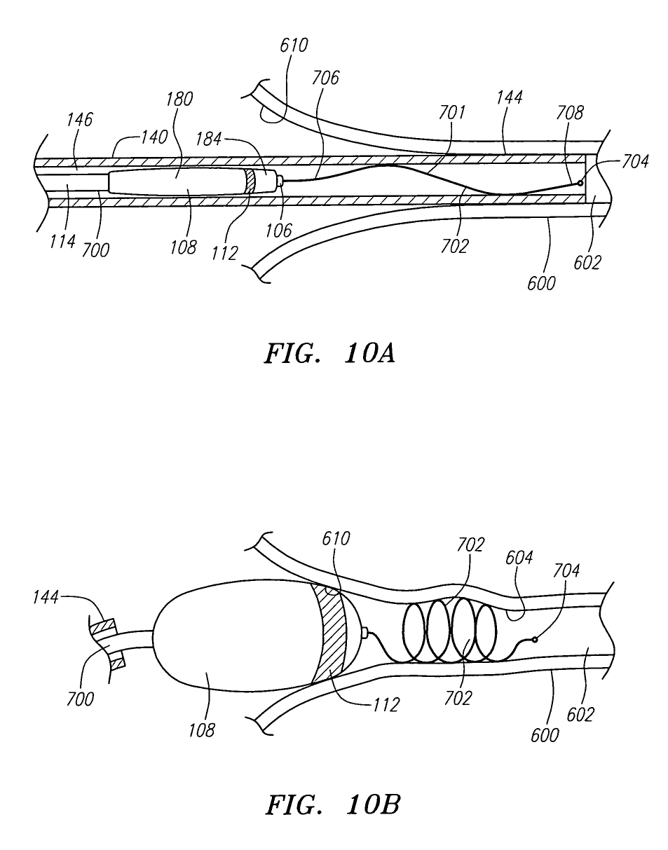

Although the above embodiments of the ablation catheter and the method have been described with reference to an ablation assembly and an anchoring device that are inflatable, the scope of the invention is not so limited. In alternative embodiments, either or both of the ablation assembly 108 and the anchoring device 110 can have other configurations that are expandable. FIGS. 10A and 10B illustrate an ablation catheter 700 having an anchoring device 701. The ablation catheter 700 is similar to the ablation catheter 102, except that the anchoring device 701 includes a wire 702 (instead of the expandable-collapsible member 170) for anchoring the ablation assembly 108. The wire 702 has a proximal end 706 secured to the distal end 106 of the shaft 114, and a distal end 708 having a blunt tip 704 for preventing injury to tissue. In other embodiments, the proximal end 706 of the wire 702 can be secured to the distal end 184 of the expandable-collapsible member 180. The wire 702 is made from an elastic material, such as nitinol, stainless steel, or plastic, such that it can be stretched to a low profile when resided within the lumen 146 of the sheath 144 (FIG. 10A). During use, the sheath 144 can be retracted relative to the ablation catheter 700 to bring the wire 702 out of the lumen 146. Outside the lumen 146, the wire 702 is unconfined and assumes an expanded configuration (FIG. 10B).

In the illustrated embodiments, the wire 702 has a helical shape when in its expanded configuration, but can also have other shapes, such as an elliptical shape or a random shape, in alternative embodiments. In its expanded configuration, the wire 702 presses against the interior wall 604 of the pulmonary vein 600 to anchor the ablation assembly 108 relative to the pulmonary vein 600.

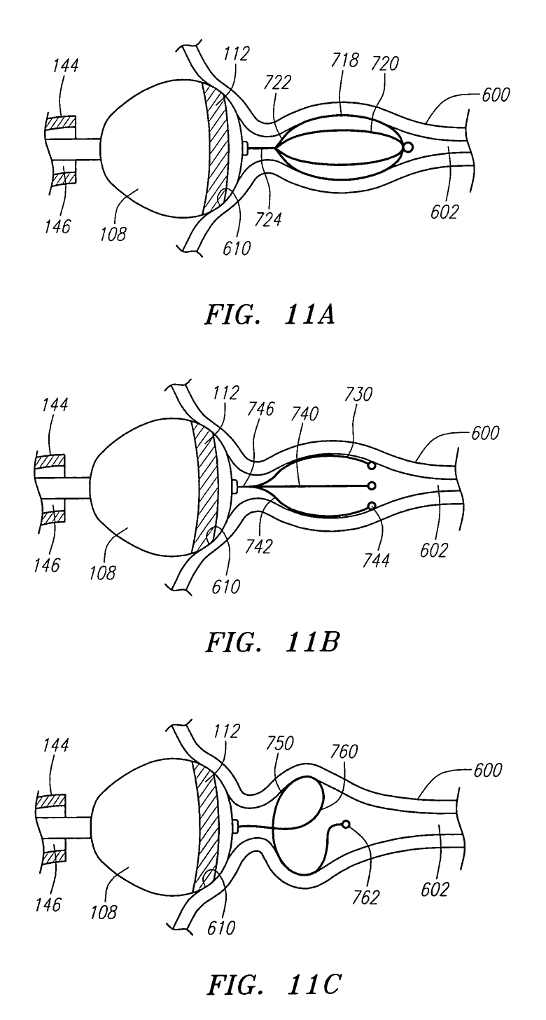

In the above described embodiments, the anchoring device 701 includes a wire 702 that has a helical shape when in its expanded configuration. However, the anchoring device 701 can also have other configurations. FIGS. 11A-11C show variations of the anchoring device that can be used instead of the wire 702. FIG. 11A shows an anchoring device 718 having a plurality of splines 720 that form a cage or basket 722. The cage 722 is secured to the distal end 106 of the shaft 114 by an elongated member 724.

Alternatively, the elongated member 724 can be secured to the ablation assembly 108. In other embodiments, the anchoring device 701 does not include the elongated member 724, and the cage 722 is secured to the ablation assembly 108. The splines 720 are made from an elastic material that allows the cage 722 to stretch to a delivery shape having a low profile when inside the sheath 144. When outside the lumen 146 of the sheath 144, the cage 722 expands to a deployed shape for anchoring the ablation assembly 108.

FIG. 11B shows an anchoring device 730 that has a plurality of wires 740 that form an assembly 742 having a fork configuration. The anchoring device 730 also includes a blunt tip 744 at the end of each of the wires 740 for preventing injury to tissue.

The assembly 742 is secured to the distal end 106 of the shaft 114 by an elongated member 746. Alternatively, the elongated member 746 can be secured to the ablation assembly 108. In other embodiments, the anchoring device 730 does not include the elongated member 746, and the assembly 742 is secured to the ablation assembly 108.

Although three wires 740 are shown, in alternative embodiments, the anchoring device 730 can have other numbers of wires 740. The wires 740 are made from an elastic material that allows the assembly 742 to stretch to a delivery shape having a low profile when inside the sheath 144. When outside the lumen 146 of the sheath 144, the assembly 742 expands to a deployed shape for anchoring the ablation assembly 108.

FIG. 11C shows an anchoring device 750, including a wire 760 that is secured to the distal end 106 of the shaft 114, and a blunt tip 762 at one end of the wire 760 for preventing injury to tissue. Alternatively, the wire 760 can be secured to the ablation assembly 108. The wire 760 is made from an elastic material that allows the wire 760 to stretch to a delivery shape having a low profile when inside the sheath 144.

When outside the lumen 146 of the sheath 144, the wire 760 forms an expanded configuration having a loop shape for anchoring the ablation assembly 108.

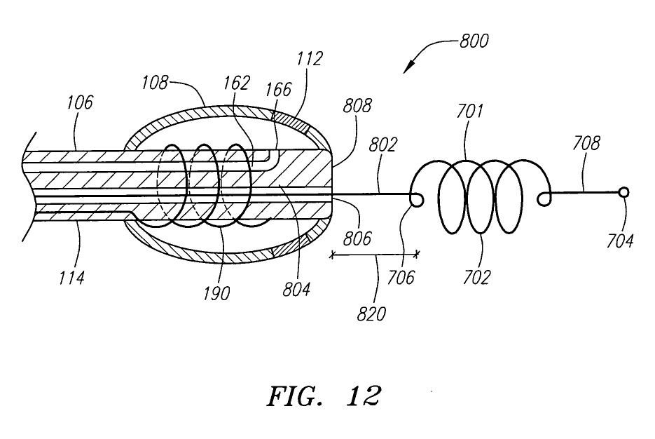

It should be noted that any of the anchoring devices described herein can be made slidable relative to the ablation assembly 108. FIG. 12 shows an ablation catheter 800 similar to the ablation catheter of FIG. 10A, except that the proximal end 706 of the anchoring device 701 is secured to an elongated member 802, such as a guide wire. In some embodiments, the elongated member 802 and the anchoring device 701 can be manufactured as a single unit. The shaft 114 further includes a lumen 804 that extends from the proximal end 104 to the distal end 106. The lumen 804 terminates at a port 806 located at a distal tip 808 of the shaft 114. The elongated member 802 is located inside the lumen 804, and can be slided relative to the shaft 114. Such configuration allows a distance 820 between the anchoring device 701 and the ablation assembly 108 be adjusted during use.

Although several examples of a catheter having an ablation assembly and an anchoring device have been described, it should be noted that the scope of the invention should not be limited to the examples described previously, and that either or both of the ablation assembly and the anchoring device can have different configurations. For example, in other embodiments, the anchoring device can include a material that swells or expands when in contact with fluid inside a body, thereby allowing the anchoring device to be secured within a pulmonary vein. Also, in other embodiments, instead of being distal to the ablation assembly, the anchoring device can be located proximal to the ablation assembly for anchoring the ablation assembly to other tissue in other applications. Further, in other embodiments, the ablation assembly can include an expandable-collapsible cage or basket that carries one or a plurality of electrodes for ablation of tissue. The cage can be made from an elastic material, such as nitinol, stainless steel, or plastic, that allows the cage to be stretched into a low profile when confined inside the lumen 146 of the sheath 140. When outside the sheath 140, the cage expands to a deployed configuration for making contact with target tissue to be ablated.

In addition, besides ablating tissue using radiofrequency energy, the ablation assembly 108 can include a transducer for applying ultrasound energy, or a fiberoptic cable for applying laser energy, to treat tissue. In other embodiments, instead of an ablation assembly 108, the catheter can include other devices for treating tissue or for sensing tissue characteristic(s). Furthermore, besides creating lesions outside the pulmonary veins, any of the embodiments of the ablation catheter described herein can be used to create lesions at other locations in the body. As such, the embodiments of the ablation catheter are not limited to treating atrial fibrillation, and can be used to treat other medical conditions.

Thus, although different embodiments have been shown and described, it would be apparent to those skilled in the art that many changes and modifications may be made thereunto without the departing from the scope of the invention, which is defined by the following claims and their equivalents.

1. A catheter, comprising:
a shaft having a distal end;
an expandable member secured to the distal end of the shaft; and
an anchoring device located adjacent to the expandable member.

2. The catheter of claim 1, the anchoring device comprising a wire.

3. The catheter of claim 2, the wire having a helical shape.

4. The catheter of claim 2, the wire forming at least one loop.

5. The catheter of claim 1, the anchoring device comprising a first wire having a proximal end, and a second wire having a proximal end, wherein the first and second wires are joined at their respective proximal ends.

6. The catheter of claim 1, the anchoring device comprising a plurality of splines.

7. The catheter of claim 1, the anchoring device comprising an expandable device.

8. The catheter of claim 7, the expandable device comprising a balloon.

9. The catheter of claim 8, the balloon, when inflated, having an elliptical shape.

10. The catheter of claim 8, the balloon, when inflated, having a pear shape.

11. The catheter of claim 8, the balloon, when inflated, having a shape that resembles an hourglass.

12. The catheter of claim 8, wherein the balloon is operative independent of the expandable member.

13. The catheter of claim 8, wherein the balloon and the expandable member are configured to be expanded simultaneously.

14. The catheter of claim 13, wherein the balloon and the expandable member have different expansion responses.

15. The catheter of claim 1, wherein the anchoring device has a delivery configuration and a deployed configuration that is different from the delivery configuration.

16. The catheter of claim 15, the anchoring device, when in its deployed configuration, having a cross sectional dimension sufficient such that the anchoring device may be secured in a pulmonary vein.

17. The catheter of claim 1, wherein the expandable member comprises a balloon.

18. The catheter of claim 17, wherein the balloon is porous.

19. The catheter of claim 17, wherein at least a portion of the balloon is electrically conductive.

20. The catheter of claim 1, wherein the expandable member comprises a plurality of splines.

21. The catheter of claim 1, the expandable member having a expanded configuration, wherein the expandable member has a cross sectional dimension that is larger than a diameter of a pulmonary vein when the expandable member is in its expanded configuration.

22. The catheter of claim 1, wherein the expandable member has a conductive region.

23. The catheter of claim 22, wherein the conductive region has a ring configuration.

24. The catheter of claim 22, wherein the conductive region is located on the expandable member such that the conductive region makes tissue contact at or adjacent an ostium of a pulmonary vein when the anchoring device is secured within the pulmonary vein.

25. The catheter of claim 1, the shaft further including a lumen extending from a proximal end of the shaft to the distal end.

26. The catheter of claim 25, wherein the lumen is sized to house a guide wire.

27. The catheter of claim 25, wherein the lumen is sized to house a steering wire.

28. The catheter of claim 1, wherein the expandable member is slidable relative to the anchoring device.

29. The catheter of claim 1, wherein the anchoring device is distal to the expandable member.

30. The catheter of claim 1, wherein the anchoring device is proximal to the expandable member.

31. An ablation catheter assembly, comprising:
a catheter having a distal end; and
an anchoring device carried in a tube at the distal end of the catheter, the anchoring device having a delivery configuration when inside the tube and a deployed configuration that is different from the delivery configuration when outside the tube, the anchoring device comprising a first wire and having a cross-sectional dimension such that the anchoring device is secured within a pulmonary vein when in its deployed configuration.

32. The catheter assembly of claim 31, the first wire having a helical shape when the anchoring device is in its deployed configuration.

33. The catheter assembly of claim 31, wherein the first wire forms at least a portion of a loop when the anchoring device is in the deployed configuration.

34. The catheter assembly of claim 31, the anchoring device further comprising a second wire.

35. The catheter assembly of claim 34, wherein a distal end of the first wire is spaced apart from a distal end of the second wire when the anchoring device is in its deployed configuration.

36. The catheter assembly of claim 31, the catheter comprising the tube.

37. The catheter assembly of claim 36, further comprising an elongated member secured to a proximal end of the anchoring device, the elongated member slidable within a lumen of the tube.

38. The catheter assembly of claim 36, wherein the first wire includes a portion which is slidable within a lumen of the tube and which extends from a proximal end of the tube to a distal end of the tube.

39. The catheter assembly of claim 31, further comprising an ablation element secured to the anchoring device.

40. The catheter assembly of claim 31, further comprising an ablation element that is slidably secured relative to the anchoring device.

41. A method of treating tissue in a body, comprising:
positioning an ablation assembly at an ostium of a body cavity;
using an anchoring device to secure the ablation assembly relative to tissue located at or adjacent the body cavity ostium; and
using the ablation assembly to deliver ablation energy to the tissue.

42. The method of claim 41, further comprising expanding the anchoring device inside the body cavity.

43. The method of claim 41, wherein the body cavity comprises a pulmonary vein.

44. The method of claim 41, wherein the ablation assembly includes a conductive region having a shape of a ring, and wherein positioning the ablation assembly is performed such that the conductive region is at or adjacent an ostium of a pulmonary vein.
